(19) United States
(12) Patent Application Publication
(10) Pub. No.: US 2014/0200241 A1
(43) Pub. Date: Jul. 17, 2014

(54) PRODRUGS FOR TREATING MICROBIAL INFECTIONS

(71) Applicants: Kim Lewis, Newton, MA (US); Laura Fleck, Lynn, MA (US); Gabriele Casadei, Bologna (IT)

(72) Inventors: Kim Lewis, Newton, MA (US); Laura Fleck, Lynn, MA (US); Gabriele Casadei, Bologna (IT)

(73) Assignee: Northeastern University, Boston, MA (US)

(21) Appl. No.: 14/119,544

(22) PCT Filed: May 24, 2012
(86) PCT No.: PCT/US12/39371

§ 371 Date: Mar. 7, 2014

Related U.S. Application Data
(60) Provisional application No. 61/489,370, filed on May 24, 2011.

(51) Int. Cl. A61K 31/47; A61K 31/12; A61K 31/345

(52) U.S. Cl. CPC A61K 31/47; A61K 31/345; A61K 31/12
USPC 514/311; 514/472; 514/689

(57) ABSTRACT

Disclosed are methods of inhibiting the growth of pathogens using prodrug compounds as described herein. Also disclosed are methods of treating microbial infections using such compounds.

This application claims priority to U.S. Provisional Patent Application No. 61/489,370, filed May 24, 2011, the contents of which are hereby incorporated by reference in their entireties.

### BACKGROUND

There is a considerable need for novel antimicrobials to combat multi-drug resistant pathogens such as yeast and bacteria. For example, Gram positive bacteria such as Clostridium difficile, which causes pseudomembranous colitis and antibiotic associated diarrhea (AAD), become resistant to antibiotics of the first- and second-line of defense, such as metronidazole and vancomycin. E. faecalis is the leading cause of hospital-acquired infections and a natural multi-drug resistant pathogen. Such bacteria are responsible for high levels of morbidity and mortality. Once resistance develops to a conventional antibiotic, modifications to the antibiotic can be introduced, but this strategy does not work very well, and switching to a novel class is a desirable goal which is extremely difficult to achieve.

MDR inhibitors have been developed for both Gram-negative and Gram-positive species. For example, a single lead compound has been identified for treating Gram-negative bacteria after screening 200,000 synthetic compounds and natural extracts. Resistance to this class of inhibitors occurred at a rate of approximately 10⁻⁹ which is equivalent to what is observed for good antibiotics. Unfortunately, prolonged administration causes nephrotoxicity. The discovery of MDR inhibitors against Gram-positive species has also been described. For example, the hit rate for an inhibitor of the NorA MDR of S. aureus from screening a compound library was 4% (Markham et al., 1999). The screen identified INF271, a compound that shows efficacy in a S. aureus mouse infection model. However, its use is also toxic.

The growing number of multidrug resistant pathogens surpasses the rate at which new antimicrobials are being put on the market. There are currently no antibiotics that sterilize infections. Therefore, there is an urgent need for novel antimicrobials to combat multi-drug resistant pathogens.

### SUMMARY OF THE INVENTION

The present disclosure is directed to prodrug compounds with potential to sterilize a broad range of pathogens. Prodrugs are able to diffuse into the cell where they are converted into a reactive compound by bacterial-specific enzymes. The activated prodrug then covalently binds to multiple targets, creating an irreversible diffusion sink within the cell.

In one aspect, the present disclosure is directed to prodrugs FL1, FL2 and PD30 having the following chemical structures:

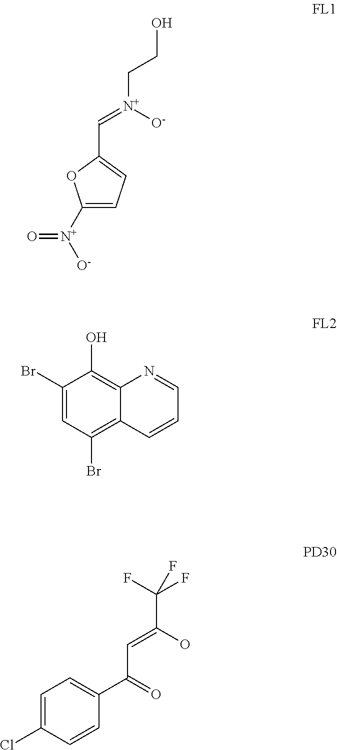 [SMILES: O=[N+]([O-])c1ccc(/C=[N+](\[O-])CCO)o1 ; Oc1c(Br)cc(Br)c2cccnc12 ; O=C(/C=C(\O)C(F)(F)F)c1ccc(Cl)cc1]

FL1 is a nitrofuratoin derivative, containing one nitrogroup (NO₂), which is a good substrate for nitroreductases. FL2 is a hydroxyquinoline derivative. Hydroxyquinolines are used as anti-protozoan and intestinal antisepsis drugs in other countries. PD30 does not belong to a known class of antimicrobials.

These and other embodiments of the invention are further described in the following sections of the application, including the Detailed Description, Examples, and Claims. Still other objects and advantages of the invention will become apparent by those of skill in the art from the disclosure herein, which are simply illustrative and not restrictive. Thus, other embodiments will be recognized by the ordinarily skilled artisan without departing from the spirit and scope of the invention.

### BRIEF DESCRIPTION OF THE FIGURES

FIG. 1 shows the kinetic curve of alamar blue reduction assay with known antimicrobials challenging E. coli at 50 μg/mL for 90 minutes (A) and S. aureus at 50 μg/mL for 120 minutes (B).

FIG. 2 shows the resazurin reduction screen for prodrugs.

FIG. 3 shows hemolytic activity of FL1, FL2, and PD30 tested using Sheep's blood.

### DETAILED DESCRIPTION

In one aspect, the present disclosure is directed to prodrugs FL1, FL2 and PD30 having the following chemical structures:

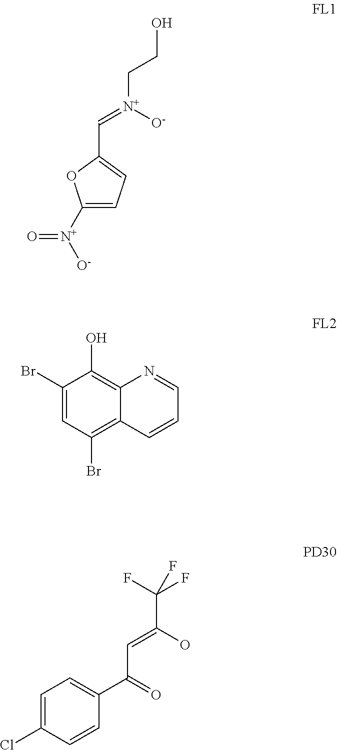 [SMILES: O=[N+]([O-])c1ccc(/C=[N+](\[O-])CCO)o1 ; Oc1c(Br)cc(Br)c2cccnc12 ; O=C(/C=C(\O)C(F)(F)F)c1ccc(Cl)cc1]

FL1 is a nitrofuratoin derivative, containing one nitrogroup (NO₂), which is a good substrate for nitroreductases. FL2 is a hydroxyquinoline derivative. Hydroxyquinolines are used as anti-protozoan and intestinal antisepsis drugs in other countries. PD30 does not belong to a known class of antimicrobials.

In some embodiments, the compound is FL1. In some embodiments, the compound is FL2. In some embodiments, the compound is PD30.

In some embodiments, the pathogen is selected from the group consisting of Escherichia sp., Bacillus sp., Yersinia sp., Francisella sp., Staphylococcus sp., Enterococcus sp., Acinetobacter sp., Pseudomonas sp., and Salmonella sp.

In some embodiments, the pathogen is selected from the group consisting of Escherichia sp., Bacillus sp., Yersinia sp., Francisella sp., Staphylococcus sp., Enterococcus sp., Acinetobacter sp., and Pseudomonas sp.

In some embodiments, the pathogen is selected from the group consisting of Escherichia sp., Bacillus sp., Staphylococcus sp., Enterococcus sp., Acinetobacter sp., and Pseudomonas sp.

In some embodiments, the pathogen is selected from the group consisting of Bacillus sp., Yersinia sp., Francisella sp., Staphylococcus sp., Enterococcus sp., Acinetobacter sp., Pseudomonas sp., and Salmonella sp.

In some embodiments, the pathogen is selected from the group consisting of Bacillus sp., Yersinia sp., Francisella sp., Staphylococcus sp., Enterococcus sp., Acinetobacter sp., and Salmonella sp.

In some embodiments, the pathogen is selected from the group consisting of Escherichia coli, Bacillus anthracis, Yersinia pestis, Francisella tularensis, Staphylococcus aureus, Enterococcus faecalis, Acinetobacter baumannii, Pseudomonas aeruginosa, and Salmonella typhimurium.

In some embodiments, the pathogen is selected from the group consisting of Escherichia coli, Bacillus anthracis, Yersinia pestis, Francisella tularensis, Staphylococcus aureus, Enterococcus faecalis, Acinetobacter baumannii, and Pseudomonas aeruginosa.

In some embodiments, the pathogen is selected from the group consisting of Escherichia coli, Bacillus anthracis, Staphylococcus aureus, Enterococcus faecalis, Acinetobacter baumannii, and Pseudomonas aeruginosa.

In some embodiments, the pathogen is selected from the group consisting of Bacillus anthracis, Yersinia pestis, Francisella tularensis, Staphylococcus aureus, Enterococcus faecalis, Acinetobacter baumannii, Pseudomonas aeruginosa, and Salmonella typhimurium.

In some embodiments, the pathogen is selected from the group consisting of Bacillus anthracis, Yersinia pestis, Francisella tularensis, Staphylococcus aureus, Enterococcus faecalis, Acinetobacter baumannii, and Salmonella typhimurium.

In some embodiments, growth of the pathogen is inhibited. In some embodiments, the pathogen is killed.

In some embodiments, the mammal is a mouse, rat, monkey, avian, dog, sheep, bovine or human. In some embodiments, the mammal is a mouse, rat, monkey, avian, sheep, or human. In some embodiments, the mammal is a mouse, avian, sheep, or human. In some embodiments, the mammal is a avian, sheep, or human. In some embodiments, the mammal is a sheep or human. In some embodiments, the mammal is a human.

It will recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Resazurin Reduction Assay

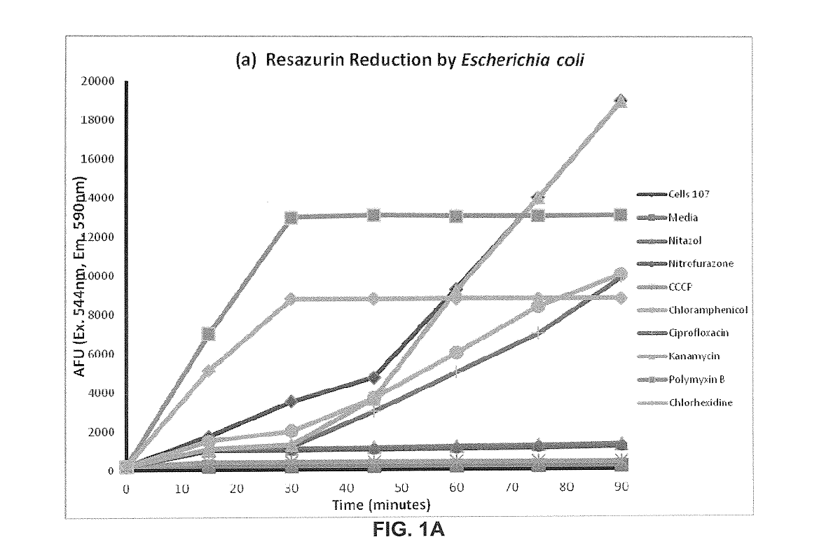

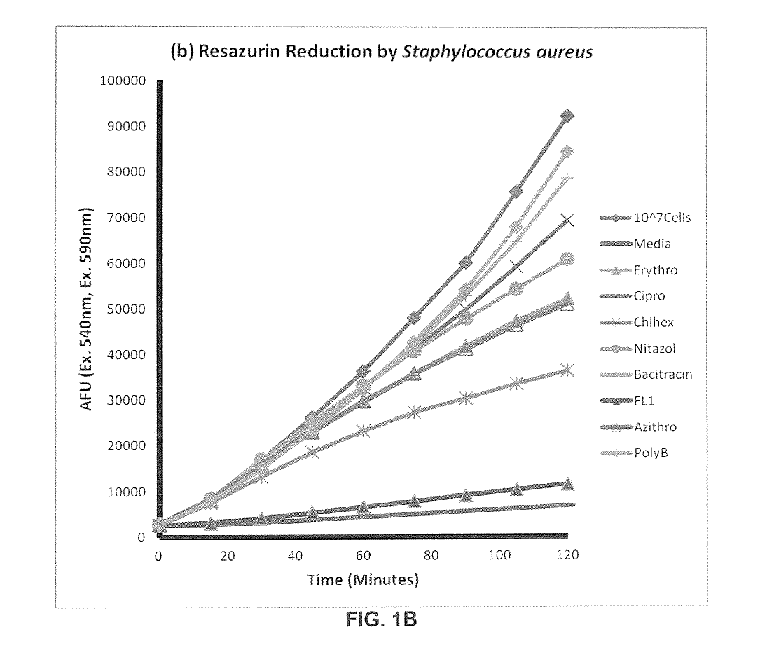

It was hypothesized that since prodrugs have multiple targets they would kill all cells and also quickly shut down metabolism. The viability dye resazurin was used to test this hypothesis and measure the metabolism of cells challenged with antiseptics, antibiotics, and the prodrug Nitazol. Different classes of antimicrobials have different mechanisms of action and consequently have diverse effects on the cells' metabolism which can be recorded using the resazurin reduction assay. Prodrugs appear to shut down metabolism quickly and produce a distinct kinetic curve in the resazurin reduction assay in Gram positive and Gram negative organisms (FIG. 1), allowing prioritization of hits. Resazurin is reduced by cells into resorufin, a fluorescent compound.

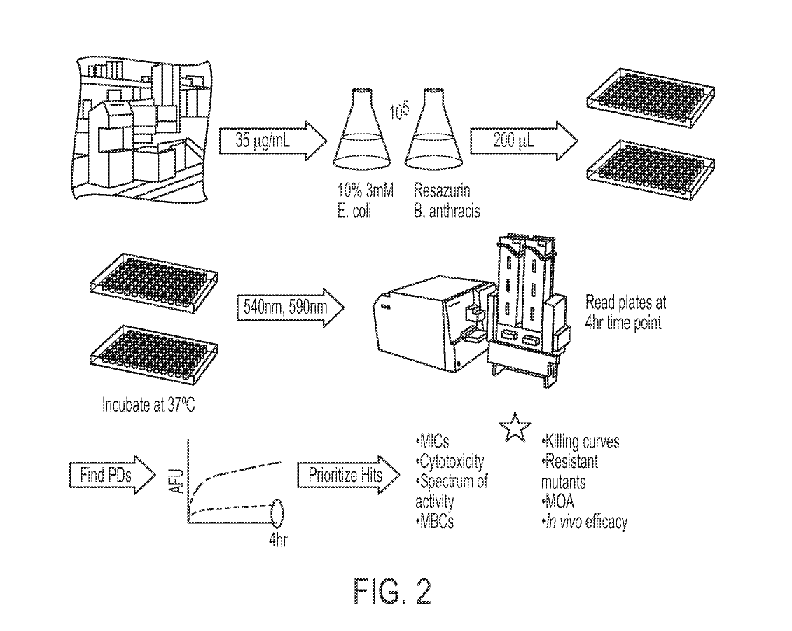

The assay was optimized for high-throughput screening (HTS). The schematic in FIG. 2 depicts the steps in the HTS. The CyBio liquid handling station was used to dry transfer compounds into 96 well plates. A total volume of 200 μL at a concentration of 10⁶ CFU/mL in Mueller Hinton Broth with 10% of a 3 mM resazurin solution was added to the screening plate and incubated at 37° C. After four hours of incubation the fluorescence was read. Compounds with an arbitrary fluorescence unit (AFU) equal to or less than that of Nitazol were considered hits and tested for their MIC, cytotoxicity, and spectrum of activity.

Three prodrug candidates were identified using this assay, named FL1, FL2 and PD30:

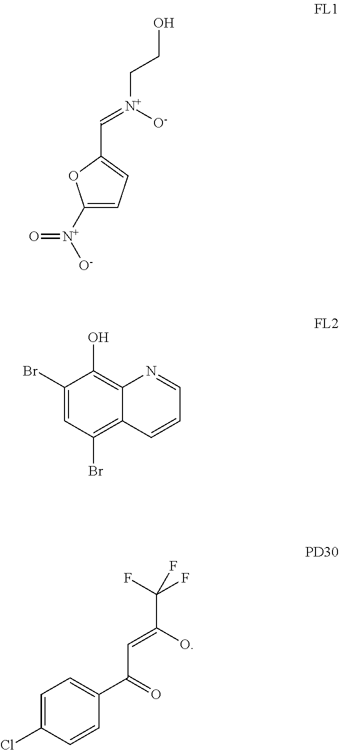

FL1 is a nitrofuratoin derivative, containing one nitrogroup (NO₂) which is good substrate for nitroreductases. FL1 has been reported, and can be synthesized according to GB 1105007, Mar. 6, 1968, herein incorporated by reference in its entirety. FL2 is a hydroxyquinoline derivative. FL2 has been reported, and can be synthesized according to Archiv der Pharmazie and Berichte der Deutschen Pharmazeutischen Gesellschaft, 1928, Vol: 266, pg. 277-80, herein incorporated by reference in its entirety. Hydroxyquinolines are used as anti-protozoan and anti-infective drugs in other countries, but have not been reported to have activity against the potential bioterrorism pathogens that we have tested. PD30 does not belong to a known class of antimicrobials. PD30 has been reported, and can be synthesized according to Journal of the Indian Chemical Society, 2003, (80)12, 1095-1101, herein incorporated by reference in its entirety.

### Example 2

### Cytotoxicity and Inhibition Assays

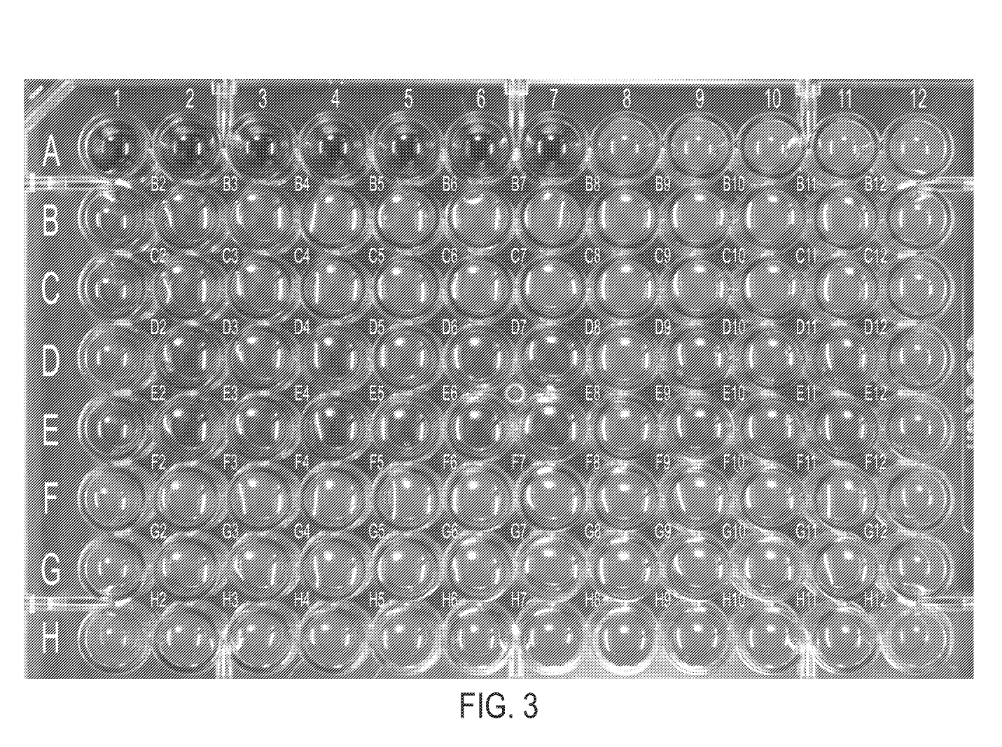

These prodrug molecules were tested for cytotoxicity in four different human cell lines, hemolysis of Sheep's red blood cells, and minimum inhibitory concentrations (MIC) against a panel of seven pathogens including Escherichia coli, Bacillus anthracis, Yersinia pestis, Francisella tularensis, Staphylococcus aureus, Enterococcus faecalis, Acinetobacter baumannii, and Pseudomonas aeruginosa. The results are shown below in Tables 1-3, and FIG. 3.

TABLE 1
Cytotoxicity and Therapeutic index (TI) for FL1, FL2, and PD30 in different cell lines.
Cytotoxicity Data

| | FL1 | | | FL2 | | | PD30 | | |
|---|---|---|---|---|---|---|---|---|---|
| | | MIC | | | MIC | | | MIC | |
| | | (μg/mL) | | | (μg/mL) | | | (μg/mL) | |
| Cell | IC50 | Against | | IC 50 | Against | | IC 50 | Against | |
| line | μg/mL | E. coli | TI | μg/mL | E. coli | TI | μg/mL | E. coli | TI |
| IMR90 | 250 | 0.78 | 320 | 100 | 1.5 | 67 | 250 | 6.25 | 40 |
| Fadu | 62.5 | 0.78 | 80 | 31.25 | 1.5 | 21 | 62.5 | 6.25 | 10 |
| HepG2 | 31.25 | 0.78 | 40 | 62.5 | 1.5 | 42 | 62.5 | 6.25 | 10 |
| Caco-2 | 200 | 0.78 | 256 | — | — | — | 200 | 6.25 | 32 |

The therapeutic index was determined by the ratio between the MIC and IC50. A therapeutic index of 10 or above is acceptable for early lead compounds.

TABLE 2
Cytotoxicity, Minimum Inhibitory Concentration and Therapeutic Index for FL1, FL2, and PD30

| | FL1 | | | FL2 | | | PD30 | | |
|---|---|---|---|---|---|---|---|---|---|
| | Cytotox | MIC | | Cytotox | MIC | | Cytotox | MIC | |
| Bacterium | (μg/mL) | (μg/mL) | TI | (μg/mL) | (μg/mL) | TI | (μg/mL) | (μg/mL) | TI |
| E. coli | 250 | 0.78 | 320 | 100 | 1.5 | 67 | 250 | 6.25 | 40 |
| B. anthracis | 250 | TBD | — | 100 | 1.5 | 67 | 250 | 0.78 | 320 |
| Y. pestis | 250 | ≦0.78 | ≧320 | 100 | 1.5 | 67 | 250 | 0.78 | 320 |
| F. tularensis | 250 | TBD | — | 100 | 0.097 | 1,031 | 250 | 0.195 | 1,282 |
| A. Baumannii | 250 | 12.5 | 20 | 100 | 6.25 | 16 | 250 | 12.5 | 20 |
| E. faecalis | 250 | 3.125 | 80 | 100 | TBD | — | 250 | 6.25 | 40 |
| S. aureus | 250 | 1.56 | 160 | 100 | TBD | — | 250 | 3.125 | 80 |
| P. aeruginosa | 250 | 25 | 10 | 100 | >50 | >2 | 250 | >50 | >2 |

Human fibroblasts were used to test for cytotoxicity. IC50 was determined, and the ratio between MIC and IC50 is the therapeutic index. Therapeutic index of 10 or above is acceptable for early lead compounds.

TABLE 3
Minimum Inhibitory Concentration (MIC) and Minimum Bactericidal
Concentration (MBC) of FL1, FL2, and PD30 against a Panel of Pathogens
Spectrum of Activity

| | FL1 | | FL2 | | PD30 | |
|---|---|---|---|---|---|---|
| | MIC | MBC | MIC | MBC | MIC | MBC |
| Species | (μg/mL) | (μg/mL) | (μg/mL) | (μg/mL) | (μg/mL) | (μg/mL) |
| Escherichia coli | 0.78 | 0.78 | 1.5 | 3 | 6.25 | |
| Bacillus anthracis | 0.35 | 0.78 | 1.5 | >50 | 0.78 | >50 |
| | (Sterne | (Sterne | (Ames | (Ames | (Ames | (Ames |
| | strain) | strain) | strain) | strain) | strain) | strain) |
| Yersinia pestis | ≦0.78 | — | 1.5 | >12.5 | >0.78 | >3.12 |
| | | | | and ≦25 | and ≦1.5 | and ≦6.25 |
| Francisella | — | — | 0.097 | >1.5 and | >0.195 | >0.78 |
| tularensis | | | | ≦3.12 | and ≦0.34 | and ≦1.5 |
| Acinetobacter | 12.5 | — | 6.25 | — | 12.5 | — |
| Baumannii | | | | | | |
| Enterococcus | 3.125 | — | 3.25 | — | 6.25 | — |
| faecalis | | | | | | |
| Staphylococcus | 1.56 | 3.25 | ≦1.5 | — | 3.12 | — |
| aureus | | | | | | |
| Pseudomonas | 25 | — | >50 | — | >50 | — |
| aeruginosa | | | | | | |
| Salmonella | 5 | — | — | — | — | — |
| typhimurium | | | | | | |

Hemolytic activity of FL1, FL2, and PD30 was tested using Sheep's blood (FIG. 3). FL1, FL2, and PD30 show no hemolytic activity at the highest concentration, 100 μg/mL (wells B1 and C1), 50 μg/mL (wells D1 and E1), and 100 μg/mL (wells F1 and G1) respectively. TritonX-100 was used as a hemolytic control with the highest concentration as 1% (Row A). Column 12 contains a PBS and PEG400 control and row H is blood alone.

### Example 3

### Converting Enzyme Assays

FL1 belongs to a well known class of compounds, the Nitrofurantoins which have one or more nitrogroups (NO₂) and are substrates of nitroreductases. We tested the activity (MIC) of FL1 against wild type E. coli and two mutants defective in the two major oxygen-insensitive nitroreductases (NfsA and NfsB). Single deletion mutations of the nitroreductases did not result in a significantly higher MIC of FL1 most probably due to compensatory expression of the alternative protein. When FL1 was tested against a double mutant, lacking both the major nitroreductases, there was a 16 fold decrease of activity (Table 4).

TABLE 4
Minimum Inhibitory Concentrations of the Nitrofuratoin Derivative,
FL1 against Strains with Deleted Potential Converting Enzymes

| E. coli Strains | MIC (μg/mL) |
|---|---|
| Wild Type | 0.78 |
| ΔnfsA/ΔnfnB | 12.5 |
| ΔnfsA | 2.5 |
| ΔnfnB | 0.16 |

FL1 has the strongest binding affinity to NfsA compared to nitazol, metronidazole, and kanamycin (Table 5). Nitazol and metronidazole are both prodrugs and converted by NfsA.

TABLE 5
FL1 has the strongest binding affintiy to NfsA compared
to nitazol, metronidazole, and kanamycin.

| Ligand | Target | Binding Affinity (kcal/mol) |
|---|---|---|
| FL1 | NfsA | −8.8 |
| Nitazol | NfsA | −4.9 |
| Metronidazole | NfsA | −4.9 |
| Kanamycin | NfsA | 18 |

FL2 and PD30 were further tested for MIC and MBC against three BSL3 pathogens F. tularensis SchuS4, Y. pestis KIM, B. anthracis Ames at the New England Regional Center of Excellence for Biodefense and Emerging Infectious Diseases (NERCE)-Harvard Medical School. The results are shown in Tables 2 and 3.

In order to identify the converting enzymes for FL2 and PD30, the compounds were tested against the wild type and a collection of “long-chromosomal-deletion” mutants supplied to the PI by the Japanese National Biological Resource Project (http://www.shigen.nig.ac.jp/ecoli/strain/nbrp/explanation/longdeletion.jsp;jsessionid=D0271E2 5866A37D05C1E21BD45B83D1A.4_{—}4). These E. coli mutants contains long chromosomal deletion 10-100 Kb, between essential genes. Once a long-deletion-mutant was able to grow in presence of 50 μg/ml of a prodrug, we identified all the deleted genes and tested the single deletion strains for resistance.

This strategy allowed us to identify a list of candidate genes for PD30, two of which also validated when tested in single-KO-mutants. The results are shown in Table 6.

Resistance to FL2 was observed in a large number of deletions, suggesting that many enzymes can potentially activate this compound. Deletions in two genes, argC and ybgJ, strongly increased resistance to PD30, suggesting that these are converting enzymes for the prodrug (Table 6).

TABLE 6
Potential converting enzymes for PD30.

| Keio Strain | MIC (μg/mL) |
|---|---|
| ΔargC | 50 |
| ΔybgJ | 100 |

The spectrum of activity for FL1 surpasses that of other nitrofuratoins that are on the market. The most unusual aspect of FL1 is its low cytotoxicity, resulting high therapeutic index, and in vivo efficacy against a MRSA infection in C. elegans. Nitrofuratoin compounds have a history of toxicity. FL1 shows a spectrum of activity and a high therapeutic index.

Hydroxyquinoline derivatives such as FL2 possess antiprotozoal, some antibacterial, and activity against C. albicans. Our findings show a spectrum of activity against biodefense organisms that have not been previously reported for this class of molecules. Former and current hydroxyquinoline compounds are not used against biodefense organisms while FL2 shows potential promise in this area.

PD30 does not belong to a known class of antimicrobials, has a broad spectrum of activity, and a high therapeutic index. PD30 could act in a new and unique way that makes it the next broad spectrum drug.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

Equivalents

Those skilled in the art will recognize, or be able to ascertain, using no more than routine experimentation, numerous equivalents to the specific substances and procedures described herein. Such equivalents are considered to be within the scope of this invention, and are covered by the following claims.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways to obtain additional embodiments within the scope and spirit of the invention.

1. A method of inhibiting pathogen growth or killing a pathogen, the method comprising contacting the pathogen with one or more compounds selected from the group consisting of: 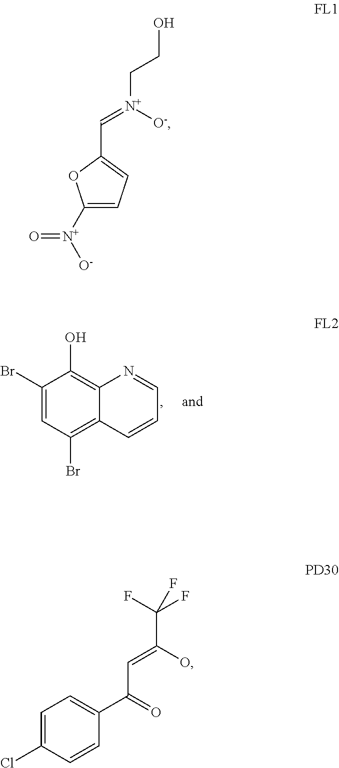
thereby inhibiting the growth of, or killing, the pathogen.

2. The method of claim 1, comprising contacting the pathogen with FL1.

3. The method of claim 1, comprising contacting the pathogen with FL2.

4. The method of claim 1, comprising contacting the pathogen with PD30.

5. The method of claim 1, wherein the pathogen is selected from the group consisting of Escherichia sp., Bacillus sp., Yersinia sp., Francisella sp., Staphylococcus sp., Enterococcus sp., Acinetobacter sp., Pseudomonas sp., and Salmonella sp.

6. The method of claim 5, wherein the pathogen is selected from the group consisting of Escherichia coli, Bacillus anthracis, Yersinia pestis, Francisella tularensis, Staphylococcus aureus, Enterococcus faecalis, Acinetobacter baumannii, Pseudomonas aeruginosa, and Salmonella typhimurium.

7. The method of claim 1, wherein pathogen growth is inhibited.

8. The method of claim 1, wherein the pathogen is killed.

9. A method of treating a microbial infection in a mammal in need thereof, the method comprising administering to the mammal a therapeutically effective amount of at least one compound selected from the group consisting of:

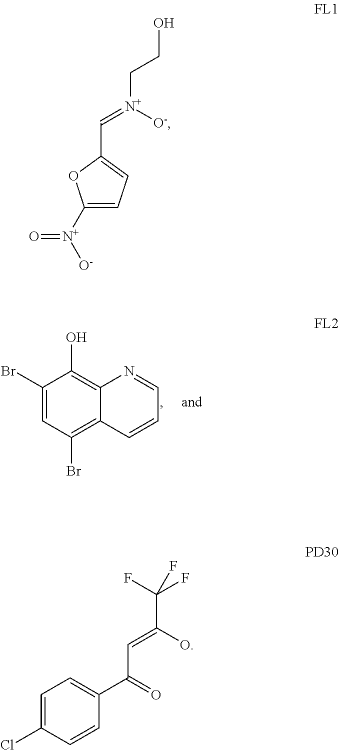

10. The method of claim 9, comprising administering FL1.

11. The method of claim 9, comprising administering FL2.

12. The method of claim 9, comprising administering PD30.

13. The method of claim 9, wherein the pathogen microbial infection is selected from the group consisting of Escherichia sp., Bacillus sp., Yersinia sp., Francisella sp., Staphylococcus sp., Enterococcus sp., Acinetobacter sp., Pseudomonas sp., and Salmonella sp. infections.

14. The method of claim 9, wherein the microbial infection is selected from the group consisting of Escherichia coli, Bacillus anthracis, Yersinia pestis, Francisella tularensis, Staphylococcus aureus, Enterococcus faecalis, Acinetobacter baumannii, Pseudomonas aeruginosa, and Salmonella typhimurium infections.

15. (canceled)

16. (canceled)

17. The method of claim 9, wherein the mammal is a sheep or human.

18. The method of claim 9, wherein the mammal is a human.
